(19) United States
(12) Patent Application Publication
(10) Pub. No.: US 2011/0243971 A1
(43) Pub. Date: Oct. 6, 2011

(54) Vaccines

(76) Inventors: Martin Friede, Cardiff, CA (US); Nathalie Garcon, Rixensart (BE)

(21) Appl. No.: 13/162,134

(22) Filed: Jun. 16, 2011

Related U.S. Application Data
(63) Continuation of application No. 09/819,464, filed on Mar. 28, 2001.
Application No. 09/819,464 is a continuation-in-part of application No. 08/945,450, filed on Dec. 12, 1997, now abandoned.
Application No. 08/945,450 is a continuation-in-part of application No. 09/269,383, filed on Apr. 2, 1999, now abandoned.

(30) Foreign Application Priority Data
Apr. 25, 1995 (GB) 9508326.7
Jun. 28, 1995 (GB) 9513107.4
Oct. 5, 1996 (GB) 9620795.6

(51) Int. Cl. A61K 39/00; A61P 37/00

(52) U.S. Cl. 424/184.1

(57) ABSTRACT

The invention relates to a vaccine composition comprising an antigen, an immunologically active saponin fraction and a sterol.

### CROSS REFERENCE TO RELATED APPLICATIONS

This is a continuation-in-part of application Ser. No. 08/945,450 filed Dec. 12, 1997, which is a 371 of International Application No. PCT/EP96/01464 filed Apr. 1, 1996, which claims priority of GB 96910019.7 filed Apr. 1, 1996, and GB 9508326.7 filed Apr. 25, 1995, and; and 09/269,383 filed Apr. 2, 1999, which is a 371 of International Application No. PCT/EP97/05578 filed Sep. 30, 1997, which claims priority of GB 9620795.6 filed Oct. 5, 1996, the contents of which are incorporated herein by reference.

### BACKGROUND OF THE INVENTION

The present invention relates to novel adjuvants, immunogenic compositions and vaccine formulations containing an immunostimulatory saponin and a sterol.

As a class, saponins are described in Lacaille-Dubois, M and Wagner H. (1996. A review of the biological and pharmacological activities of saponins. Phytomedicine vol 2 pp 363-386). Saponins are steroid or triterpene glycosides widely distributed in the plant and marine animal kingdoms. Saponins are noted for forming colloidal solutions in water which foam on shaking, and for precipitating cholesterol. When saponins are near cell membranes they create pore-like structures in the membrane which cause the membrane to burst. Haemolysis of erythrocytes is an example of this phenomenon, which is a property of certain, but not all, saponins.

Saponins are known as adjuvants in vaccines for systemic administration. The adjuvant and haemolytic activity of individual saponins has been extensively studied in the art (Lacaille-Dubois and Wagner, supra). For example, Quil A (derived from the bark of the South American tree Quillaja Saponaria Molina), and fractions thereof, are described in U.S. Pat. No. 5,057,540 and “Saponins as vaccine adjuvants”, Kensil, C. R., Crit Rev Ther Drug Carrier Syst, 1996, 12 (1-2):1-55; and EP 0 362 279 B1. Quillaia saponin has also been disclosed as an adjuvant by Scott et al, Int. Archs. Allergy Appl. Immun., 1985, 77, 409. QuilA and cholesterol containing liposomes are described in Lipford et al., 1994, Vaccine, 12, 1, 73-80. Quil A immunogenic compositions are also described in Bomford, 1980, Int. Archs. Allergy appl. Immun., 63, 170-177; Bomford, 1982, Int. Archs. Allergy appl. Immun., 67, 127-131; Scott et al., 1985, Int. Archs. Allergy appl. Immun., 77, 409-412.

Particulate structures, termed Immune Stimulating Complexes (ISCOMS), comprising Quil A are haemolytic and have been used in the manufacture of vaccines (Morein, B., EP 0 109 942 B1; WO 96/11711; WO 96/33739). The haemolytic saponins QS21 and QS17 (HPLC purified fractions of Quil A) have been described as potent systemic adjuvants, and the method of their production is disclosed in U.S. Pat. No. 5,057,540 and EP 0 362 279 B1. It has been described that using conventional techniques that formation of ISCOMs with QS21 alone is not possible (WO 92/06710, page 19, table D), the techniques of the present invention make it possible to formulate QS21 into ISCOM structures. Oil emulsions comprising Quil A have also been described, wherein the QuilA may be complexed with a sterol (U.S. Pat. No. 4,806,350).

Other saponins which have been used in systemic vaccination studies include those derived from other plant species such as Gypsophila and Saponaria (Bomford et al., Vaccine, 10(9):572-577, 1992). However, use of saponins, and in particular QS21, as adjuvants is associated with a number of disadvantages. For example when QS21 is injected into a mammal as a free molecule it has been observed that necrosis, that is to say, localised tissue death, occurs at the injection site. In addition, it has been shown for some isolated saponins, including pure QS21, are difficult to formulate in particulate structures in which the saponin is in a stable form.

### BRIEF SUMMARY OF THE INVENTION

The present invention relates to novel adjuvants, immunogenic compositions and vaccine formulations. In particular, the present invention relates to adjuvants which contain an immunostimulatory saponin and a sterol. Particularly preferred saponin fractions are those derived from the bark of Quillaja Saponaria Molina, and more particularly those which are isolated as an HPLC peak, such as QS21, and the preferred sterol is cholesterol. The adjuvants of the present invention may be in a particulate form, and may be formulated with a carrier, and in a preferred embodiment of the present invention the carrier is a metallic salt particle, such as aluminium hydroxide or aluminium phosphate. Immunogenic compositions and vaccines which comprise the adjuvants of the present invention and at least one antigen are provided. Additionally provided are methods for the production of the adjuvant and vaccine formulations, their use in medicine and in the prophylaxis and therapy of disease.

### BRIEF DESCRIPTION OF THE DRAWINGS

FIG. 1 is a graph showing a comparison of QS21 quenching by liposomes containing or lacking cholesterol.

FIG. 2 is a graph showing hydrosysis of QS21 in alkaline aqueous medium.

FIG. 3 is a graph showing anti-gp120 CTL activity generated by QS21 as an adjuvant.

FIG. 4 is a graph showing anti-gp120 CTL activity generated by QS21 and cholesterol containing lipsome as adjuvant.

FIG. 5 is a graph showing anti-gD antibodies in AGM.

FIG. 6 is a graph showing that antigen specific proliferation was measured by stimulation in vitro with gD coupled to microbeads, and expressed as CPM of 3H-TdR incorporated.

FIG. 7 is a graph showing IL-2 production of cells after gD vaccination and restimulation in vitro.

FIG. 8 is a graph showing interferon gamma production of cells after gD vaccination and restimulation in vitro.

FIG. 9 is a graph showing RSV neutralisation titres and anti FG ELISA titres after vaccination.

FIG. 10 is a graph showing the comparison of QS21-SUV containing formulations with Alum formulation kinetics of the anti-HBs response (post I/II).

FIG. 11 is a graph showing the comparison of QS21-SUV containing formulations with Alum formulation isotypic profile (post anti-HBs response.

### DETAILED DESCRIPTION OF THE INVENTION

It has now surprisingly been found that many of the problems of saponin adjuvants can be overcome by the present invention, for example, necrosis at the injection site can be avoided by use of formulations containing a combination of an immunologically active saponin and a sterol. In addition, certain adjuvant formulations, such as those that comprise QS21 and a sterol, are more stable in that the saponin is stabilised against base mediated hydrolysis. Additionally, the adjuvant compositions of the present invention are extremely potent in the induction of cell mediated immune responses, including cytolytic T-cell responses. The present invention therefore provides adjuvant compositions, immunogenic compositions and vaccine compositions comprising an immunologically active saponin fraction and a sterol. The preferred immunologically active saponin fractions are those which may be derived from the bark of Quillaja Saponaria Molina. In particular the saponin fractions are derived from the bark of Quillaja Saponaria Molina as a single HPLC peak. In the case of immunogenic compositions and the vaccines of the present invention, the formulations further comprise at least one antigen.

Preferred sterols for use in the adjuvant compositions of the present invention include β-sitosterol, stigmasterol, ergosterol, ergocalciferol and cholesterol. These sterols are well known in the art, for example cholesterol is disclosed in the Merck Index, 11th Edn., page 341, as a naturally occurring sterol found in animal fat. Most preferably the sterol is cholesterol.

Preferably the compositions of the invention contain an immunologically active saponin fraction from the bark of Quillaja Saponaria molina in substantially purified form. “Purified saponin” is intended to mean a substantially pure saponin which is purified to one or more of to following standards: 1) appearing as only one major carbohydrate staining band on silica gel TLC (EM Science HPTLC Si60) in a solvent system of 40 mM acetic acid in chloroform/methanol/water (60/45/10 v/v/v); 2) appearing as only one major carbohydrate staining band on reverse phase TLC (EM Science Silica Gel RP-8) in a solvent system of methanol/water (70/30 v/v); or 3) appearing as only one major peak upon reverse phase HPLC on a vydac C4 (5 micrometer particle size, 300 angstrom pore size, 4.6 mm ID×25 cm L) in 40 mM acetic acid in methanol/water (58/42 v/v). Preferably the compositions of the invention contain the saponin fraction QS21. The QS21 is preferably in a substantially purified form, that is to say, as isolated by collection of a single HPLC peak after the separation of a saponin from the bark of Quillaja saponaria molina, or more specifically the QS21 is at least 90% pure, preferably at least 95% pure and most preferably at least 98% pure.

Other immunologically active saponin fractions useful in compositions of the invention include QA17/QS17. Compositions of the invention comprising QS21 and cholesterol show decreased reactogenicity when compared to compositions in which the cholesterol is absent, while the adjuvant effect is maintained. In addition it is known that pure QS21 degrades under basic conditions where the pH is about 7 or greater. A further advantage of the present compositions is that the stability of pure QS21 to base-mediated hydrolysis is enhanced in formulations containing cholesterol. Accordingly, there is provided an adjuvant formulation comprising a purified and stable QS21 saponin which is substantially devoid of hydrolysed QS21, as detected by HPLC.

The ratio of saponin:sterol in the adjuvant formulations will typically be in the order of 1:100 to 5:1 weight to weight. However, when the adjuvant formulation is in the form of an ISCOM, the saponin must be QS21. More preferably, excess sterol is present, and more preferably the ratio of saponin:sterol is at least 1:2 w/w, and most preferably the ratio will be 1:5 (w/w).

In a preferred embodiment, when the saponin is QS21, the ratio of QS21 contained within the saponin fraction sterol will typically be in the order of 1:100 to 1:1 weight to weight. Preferably excess sterol to QS21 is present, and more preferably the ratio of QS21:sterol being at least 1:2 w/w, and most preferably the ratio will be 1:5 (w/w).

Preferred adjuvants of the present invention comprise the saponin and the sterol in a vesicle-like structure. In particular, preferred adjuvants of the present invention are those forming a liposome. Compositions where the sterol/immunologically active saponin fraction forms an ISCOM structure also form an aspect of the invention, when the saponin is QS21.

In these vesicular embodiments of the present invention the adjuvant formulation preferably further comprises a lipid capable of forming a bilayer membrane. Accordingly, the liposomes or ISCOMs preferably contain a neutral lipid, for example phosphatidylcholine, which is preferably non-crystalline at room temperature, for example egg yolk phosphatidylcholine, dioleoyl phosphatidylcholine or dilauryl phosphatidylcholine, and of these lipids dioleoyl phosphatidylcholine is most preferred. The vesicles may also contain a charged lipid which increases the stability of the liposome-QS21 structure for liposomes composed of saturated lipids. In these cases the amount of charged lipid is preferably 1-20% w/w, most preferably 5-10%. The ratio of sterol to phospholipid is 1-50% (mol/mol), most preferably 20-25%. Typically, if both are present, the sterol (cholesterol):phosphatidylcholine ratio is (1:4 w/w).

The vesicular adjuvants of the present invention may be unilamellar or multilamellar. Most preferably the vesicles are unilamellar liposomes. The size of the vesicles are typically in the range of 10-1000 nm (mean particle size) and more preferably between 10-220 nm, and most preferably between 10-150 nm in size such as around 115 nm. Accordingly small unilamellar vesicles (SUV) with a mean diameter particle size of between 70-150 nm comprising the saponin and the sterol (preferably QS21 and cholesterol) where there is excess sterol present are particularly preferred adjuvants of the present invention.

In these vesicular adjuvants of the present invention the ratio of the sterol to the saponin is important in determining the structure of the adjuvant. Accordingly, the liposomal adjuvants comprise excess sterol to the saponin and will typically be in the order of 1:100 to 1:1 weight to weight, and most preferably the ratio of saponin:sterol being at least 1:2 w/w, and most preferably the ratio will be 1:5 (w/w). ISCOM structure adjuvants of the present invention typically have excess saponin to sterol, and preferably the ratio of saponin:sterol will be 5:1 weight to weight (w/w). For these vesicular embodiments of the present invention QS21 is the preferred saponin and cholesterol is the preferred sterol, and the above ratios apply to these molecules accordingly.

Typically for human administration saponin and sterol will be present in a vaccine in the range of about 1 μg to about 100 μg, preferably about 10 μg to about 50 μg per dose.

In an related aspect of the present invention, there is provided an adjuvant composition comprising a tripartite combination of a saponin (such as a fraction of Quillaja saponaria bark), a sterol, and a derivative of LPS. The most preferred adjuvant combination is QS21, 3D-MPL and cholesterol.

It has long been known that enterobacterial lipopolysaccharide (LPS) is a potent stimulator of the immune system, although its use in adjuvants has been curtailed by its toxic effects. A non-toxic derivative of LPS, monophosphoryl lipid A (MPL), produced by removal of the core carbohydrate group and the phosphate from the reducing-end glucosamine, has been described by Ribi et al (1986, Immunology and Immunopharmacology of bacterial endotoxins, Plenum Publ. Corp., NY, p 407-419) and has the following structure:

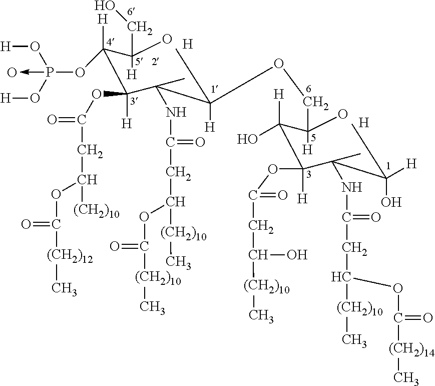

A further detoxified version of MPL results from the removal of the acyl chain from the 3-position of the disaccharide backbone, and is called 3-O-Deacylated monophosphoryl lipid A (3D-MPL). It can be purified and prepared by the methods taught in GB 2122204B, which reference also discloses the preparation of diphosphoryl lipid A, and 3-O-deacylated variants thereof. A preferred form of 3D-MPL is in the form of an emulsion having a small particle size less than 0.2 μm in diameter, and its method of manufacture is disclosed in WO 94/21292. Aqueous formulations comprising monophosphoryl lipid A and a surfactant have been described in WO 98/43670A2.

The bacterial lipopolysaccharide derived adjuvants to be formulated in the adjuvants of the present invention may be purified and processed from bacterial sources, or alternatively they may be synthetic. For example, purified monophosphoryl lipid A is described in Ribi et al 1986 (supra), and 3-O-Deacylated monophosphoryl or diphosphoryl lipid A derived from Salmonella sp. is described in GB 2220211 and U.S. Pat. No. 4,912,094. Other purified and synthetic lipopolysaccharides have been described (U.S. Pat. No. 6,005,099 and EP 0 729 473 B1; Hilgers et al., 1986, Int. Arch. Allergy. Immunol., 79(4):392-6; Hilgers et al., 1987, Immunology, 60(1):141-6; and EP 0 549 074 B1). Particularly preferred bacterial lipopolysaccharide adjuvants are 3D-MPL and the β(1-6) glucosamine disaccharides described in U.S. Pat. No. 6,005,099 and EP 0 729 473 B1.

Accordingly, the LPS derivatives that may be used in the present invention are those immunostimulants that are similar in structure to that of LPS or MPL or 3D-MPL. In another aspect of the present invention the LPS derivatives may be an acylated monosaccharide, which is a sub-portion to the above structure of MPL.

A preferred disaccharide LPS derivative adjuvant, is a purified or synthetic lipid A of the following formula:

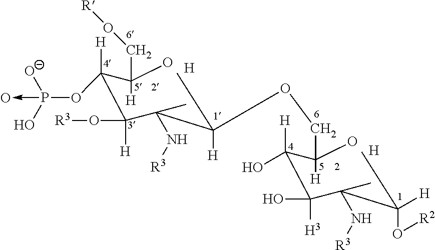

wherein R2 may be H or PO3H2; R3 may be an acyl chain or β-hydroxymyristoyl or a 3-acyloxyacyl residue having the formula:

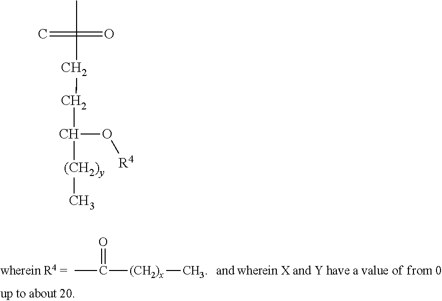

The LPS derivative may be formulated with the saponin containing structures or may be simply admixed with the saponin containing structures. Suitable compositions of the invention are those wherein the sterol/saponin containing liposomes or ISCOMs are initially prepared without the LPS derivative, and the LPS derivative is then added, preferably as particles with an average diameter of about 100 nm. In these embodiments the LPS derivative is therefore not contained within the vesicle membrane (known as LPS derivative-out). Compositions where an LPS derivative is contained within the vesicle membrane (known as LPS derivative-in) also form an aspect of the invention. In this regard the adjuvant formulations preferably comprise a sterol and saponin containing liposome, and the LPS derivative (preferably 3D-MPL) is contained within the liposome membrane. Adjuvant formulations comprising 3D-MPL in the membrane of a liposomal formulation are particularly potent in the induction of cell mediated immune responses, and form an alternative aspect of the present invention.

The antigen can be contained within the vesicle membrane or contained outside the vesicle membrane. Preferably soluble antigens are outside and hydrophobic or lipidated antigens are either contained inside or outside the membrane.

Often the adjuvants of the invention will not require any specific carrier and be formulated in an aqueous or other pharmaceutically acceptable buffer. In some cases it may be advantageous that the vaccines of the present invention will further contain a carrier such as a metallic salt particle, or be presented in an oil in water emulsion, or other suitable vehicle, such as for example, additional liposomes, microspheres or encapsulated antigen particles.

Particularly preferred adjuvants of the present invention comprise a saponin, a sterol and a metallic salt particle carrier. Examples of metallic salt particles which may be used in formulating the adjuvants of the present invention include salts of aluminium, zinc, calcium, cerium, chromium, iron, or berilium. Preferred salts of these metals are phosphate or hydroxide salts. Particularly preferred metallic salt carriers are the aluminium salts aluminium hydroxide or aluminium phosphate (Gupta, R., 1998, Advanced Drug Delivery Reviews, 32, 155-172).

Incorporation of aluminium salts in vaccine formulations containing an LPS derivative, a saponin fraction (such as QS21) and cholesterol containing SUV enhances both humoral and cellular responses and that vaccine formulations containing 3D-MPL, QS21, SUV and alum are non-toxic with a good reactogenicity profile and have enhanced adjuvant activity. In addition, the combined adjuvant appears to favour TH1 responses. In this regard, a preferred adjuvant formulation comprises QS21 and cholesterol containing SUV, adsorbed onto an aluminium salts, such as aluminium hydroxide or aluminium phosphate. A further enhancement of this adjuvant formulation can be obtained by the addition of 3D-MPL into the lipid bilayer.

The adjuvants of the present invention may be manufactured using techniques known in the art. For example, the saponin and cholesterol may be admixed in a suitable detergent, followed by a solvent extraction technique to form the liposomes or ISCOMs of the present invention.

However, the present inventors have developed a process of manufacture that in itself has several advantages over the known methods. The preferred process by which the adjuvants of the present invention involves the manufacture of small unilamellar liposomes (SUV) comprising a sterol such as cholesterol, to which the saponin is admixed. For example a sample of cholesterol containing SUV (cSUV) may be added to QS21 at a ratio of 5:1 (cholesterol:QS21 w/w), which results in the QS21 associating with the liposomal bilayer membrane, which results in the formation of a liposomal structure. Alternatively, the cSUV may be added to the QS21 at a ratio of 1:5 (cholesterol:QS21 w/w), which results in the QS21 associating with the liposomal bilayer membrane and creating a “cage-like” ISCOM structure.

In a preferred aspect of the invention, liposomes/SUV are first added to the QS21 and then mixed with alum which results in a significant proportion of the QS21 binding to the alum (via interaction through the liposomes). Such a formulation, when injected, is expected to result in a slower release of QS21 to the body, due to a depot effect of the alum, than if the QS21 was free or in un-fixed liposomes. The formulation containing 3D-MPL, QS21, SUV and alum are particularly advantageous as they are non-toxic and highly immunogenic.

Phosphate buffered saline may be used as the aqueous buffer medium, the pH of the buffer may be neutral or slightly alkaline or slightly acidic. Accordingly, the pH may be in a range of pH 6 to 8. The strength of the buffer may be between 10-50 mM PO₄ and between 10-150 mM. If an LPS derivative is present in the adjuvant formulation the pH is preferably slightly acidic, and most preferably is around pH 6.1.

In a related aspect of the present invention there is provided a method of stabilising an adjuvant formulation comprising a derivative of LPS (and in particular 3D-MPL) in a vesicle membrane, by formulating the adjuvant composition at around pH 6.1.

The present invention provides an immunogenic composition or vaccine composition comprising a metallic salt particle such as aluminium hydroxide or aluminium phosphate, an antigen, an immunologically active saponin fraction and a sterol.

Preferably the vaccine formulations will contain the adjuvant compositions of the present invention and an antigen or antigenic composition capable of eliciting an immune response against a human or animal pathogen. Antigen or antigenic compositions known in the art can be used in the compositions of the invention, including polysaccharide antigens, antigen or antigenic compositions derived from HIV-1, (such as gp120 or gp160), any of Feline Immunodeficiency virus, human or animal herpes viruses, such as gD or derivatives thereof or Immediate Early protein such as ICP27 from HSV1 or HSV2, cytomegalovirus (especially human) (such as gB or derivatives thereof), Varicella Zoster Virus (such as gpl, II or III), or from a hepatitis virus such as hepatitis B virus for example Hepatitis B Surface antigen or a derivative thereof, hepatitis A virus, hepatitis C virus and hepatitis E virus, or from other viral pathogens, such as Respiratory Syncytial virus (for example HSRV F and G proteins or immunogenic fragments thereof disclosed in U.S. Pat. No. 5,149,650 or chimeric polypeptides containing immunogenic fragments from HSRV proteins F and G, eg FG glycoprotein disclosed in U.S. Pat. No. 5,194,595), antigens derived from meningitis strains such as meningitis A, B and C, Streptococcus Pneumonia, human papilloma virus, Influenza virus, Haemophilus Influenza B (Hib), Epstein Barr Virus (EBV), or derived from bacterial pathogens such as Salmonella, Neisseria, Borrelia (for example OspA or OspB or derivatives thereof), or Chlamydia, or Bordetella for example P.69, PT and FHA, or derived from parasites such as plasmodium or toxoplasma.

HSV Glycoprotein D (gD) or derivatives thereof is a preferred vaccine antigen. It is located on the viral membrane, and is also found in the cytoplasm of infected cells (Eisenberg R. J. et al; J of Virol 1980 35 428-435). It comprises 393 amino acids including a signal peptide and has a molecular weight of approximately 60 kD. Of all the HSV envelope glycoproteins this is probably the best characterised (Cohen et al J. Virology 60 157-166). In vivo it is known to play a central role in viral attachment to cell membranes. Moreover, glycoprotein D has been shown to be able to elicit neutralising antibodies in vivo and protect animals from lethal challenge. A truncated form of the gD molecule is devoid of the C terminal anchor region and can be produced in mammalian cells as a soluble protein which is exported into the cell culture supernatant. Such soluble forms of gD are preferred. The production of truncated forms of gD is described in EP 0 139 417. Preferably the gD is derived from HSV-2. An embodiment of the invention is a truncated HSV-2 glycoprotein D of 308 amino acids which comprises amino acids 1 through 306 naturally occurring glycoprotein with the addition Asparagine and Glutamine at the C terminal end of the truncated protein devoid of its membrane anchor region. This form of the protein includes the signal peptide which is cleaved to allow for the mature soluble 283 amino acid protein to be secreted from a host cell.

In another aspect of the invention, Hepatitis B surface antigen is a preferred vaccine antigen. As used herein the expression ‘Hepatitis B surface antigen’ or ‘HBsAg’ includes any HBsAg antigen or fragment thereof displaying the antigenicity of HBV surface antigen. It will be understood that in addition to the 226 amino acid sequence of the HBsAg antigen (see Tiollais et al, Nature, 317, 489 (1985) and references therein) HBsAg as herein described may, if desired, contain all or part of a pre-S sequence as described in the above references and in EP-A-0 278 940. In particular the HBsAg may comprise a polypeptide comprising an amino acid sequence comprising residues 12-52 followed by residues 133-145 followed by residues 175-400 of the L-protein of HBsAg relative to the open reading frame on a Hepatitis B virus of ad serotype (this polypeptide is referred to as L*; see EP 0 414 374). HBsAg within the scope of the invention may also include the pre-S1-preS2-S polypeptide described in EP 0 198 474 (Endotronics) or close analogues thereof such as those described in EP 0 304 578 (Mc Cormick and Jones). HBsAg as herein described can also refer to mutants, for example the ‘escape mutant’ described in WO 91/14703 or European Patent Application Number 0 511 855A1, especially HBsAg wherein the amino acid substitution at position 145 is to arginine from glycine.

Normally the HBsAg will be in particle form. The particles may comprise for example S protein alone or may be composite particles, for example (L*,S) where L* is as defined above and S denotes the S-protein of HBsAg. The said particle is advantageously in the form in which it is expressed in yeast.

The preparation of hepatitis B surface antigen S-protein is well documented. See for example, Harford et al (1983) in Develop. Biol. Standard 54, page 125, Gregg et al (1987) in Biotechnology, 5, page 479, EP 0 226 846, EP 0 299 108 and references therein.

In another embodiment, the vaccine antigen is an RSV antigen. In particular an F/G antigen. U.S. Pat. No. 5,194,595 (Upjohn) describes chimeric glycoproteins containing immunogenic segments of the F and G glycoproteins of RSV and suggests that such proteins can be expressed from a variety of systems including bacterial, yeast, mammalian (eg CHO cells) and insect cells (using for example a baculovirus).

Wathen et al (J. Gen. Virol. 1989, 70, 2625-2635) describes a particular RSV FG chimeric glycoprotein expressed using a baculovirus vector consisting of amino acids 1-489 of the F protein linked to amino acids 97-279 of the G protein. The formulations within the scope of the invention may also contain an anti-tumour antigen and be useful for immunotherapeutically treating cancers.

Vaccine preparation is generally described in New Trends and Developments in Vaccines, edited by Voller et al., University Park Press, Baltimore, Md., U.S.A. 1978. Encapsulation within liposomes is described, for example, by Fullerton, U.S. Pat. No. 4,235,877. Conjugation of proteins to macromolecules is disclosed, for example, by Likhite, U.S. Pat. No. 4,372,945 and by Armor et al., U.S. Pat. No. 4,474,757.

The amount of protein in each vaccine dose is selected as an amount which induces an immunoprotective response without significant, adverse side effects in typical vaccinees. Such amount will vary depending upon which specific immunogen is employed and how it is presented. Generally, it is expected that each dose will comprise 1-1000 μg of protein, preferably 2-100 μg, most preferably 4-40 μg. An optimal amount for a particular vaccine can be ascertained by standard studies involving observation of appropriate immune responses in subjects. Following an initial vaccination, subjects may receive one or several booster immunisation adequately spaced.

The formulations of the present invention maybe used for both prophylatic and therapeutic purposes. Accordingly in a further aspect, the invention therefore provides use of a vaccine of the invention for the treatment of human patients. The invention provides a method of treatment comprising administering an effective amount of a vaccine of the present invention to a patient. In particular, the invention provides a method of treating viral, bacterial, parasitic infections or cancer which comprises administering an effective amount of a vaccine of the present invention to a patient.

In alternative aspects of the present invention there is provided methods of reducing the reactogenicity of QS21 containing adjuvant formulations, and also a method of stabilising QS21 against alkali mediated hydrolysis, by the addition of excess sterol (particularly cholesterol) to the adjuvant formulation (weight/weight).

The following examples and data illustrates the invention.

### EXAMPLES

### 1.1 Method of Preparation of Liposomes

A mixture of lipid (such as phosphatidylcholine either from egg-yolk or synthetic) and cholesterol in organic solvent, is dried down under vacuum (or alternatively under a stream of inert gas). An aqueous solution (such as phosphate buffered saline) is then added, and the vessel agitated until all the lipid is in suspension. This suspension is then microfluidised until the liposome size is reduced to 100 nm, and then sterile filtered through a 0.2 μm filter. Extrusion or sonication could replace this step.

Typically the cholesterol:phosphatidylcholine ratio is 1:4 (w/w), and the aqueous solution is added to give a final cholesterol concentration of 5 to 50 mg/ml. If 3D-MPL in organic solution is added to the lipid in organic solution the final liposomes contain 3D-MPL in the membrane (referred to as 3D-MPL in).

The liposomes have a defined size of 100 nm and are referred to as SUV (for small unilamelar vesicles). If this solution is repeatedly frozen and thawed the vesicles fuse to form large multilamellar structures (MLV) of size ranging from 500 nm to 15 μm.

The liposomes by themselves are stable over time and have no fusogenic capacity.

### 1.2 Formulation Procedure

QS21 in aqueous solution is added to the liposomes. This mixture is then added to the antigen solution which may if desired contain 3D-MPL in the form of 100 nm particles.

### 1.3 The Lytic Activity of QS21 is Inhibited by Liposomes Containing Cholesterol

When QS21 is added to erythrocytes, they lyse them releasing hemoglobin. This lytic activity can also be measured using liposomes which contain cholesterol in their membrane and an entrapped fluorescent dye, carboxyfluorescein—as the liposomes are lysed the dye is released which can be monitored by fluorescence spectroscopy. If the fluorescent liposomes do not contain cholesterol in their membrane no lysis of the liposomes is observed.

If the QS21 is incubated with liposomes containing cholesterol prior to adding it to erythrocytes, the lysis of the erythrocytes is diminished depending on the ratio of cholesterol to QS21. If a 1:1 ratio is used no lytic activity is detected. If the liposomes do not contain cholesterol, inhibition of lysis requires a one thousand fold excess of phospholipid over QS21.

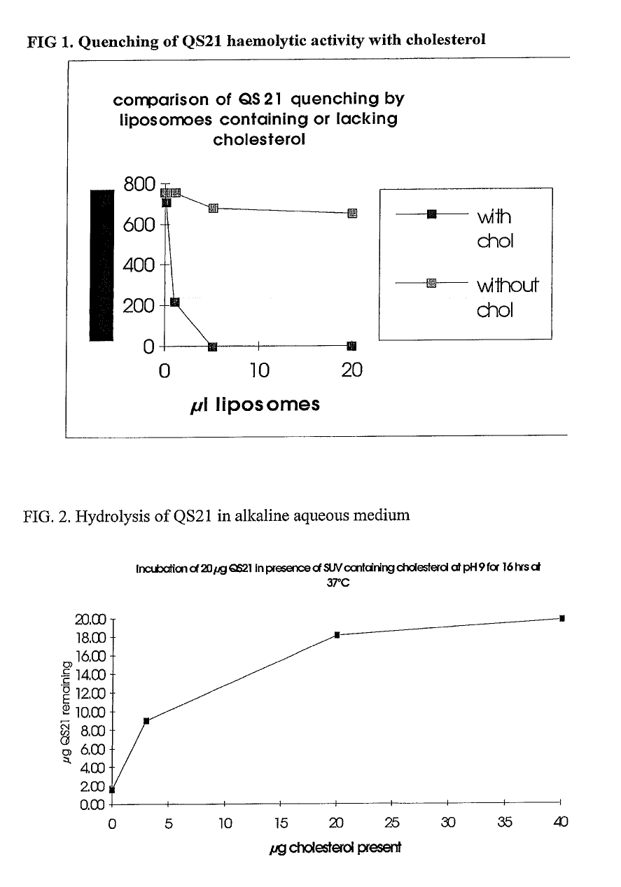

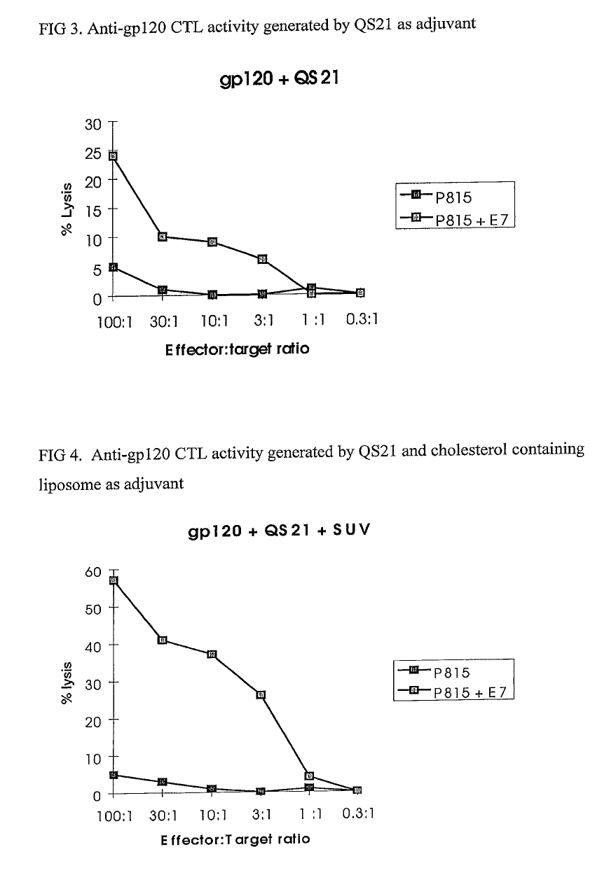

The same holds true using fluorescent liposomes to measure the lytic activity. In FIG. 1, the lytic activity of 4 μg of QS21 treated with liposomes lacking cholesterol (1 mg egg yolk lecithin per ml) or containing cholesterol (1 mg lecithin, 500 μg cholesterol per ml) was measured by fluorescence.

The data shows that QS21 associates in a specific manner with cholesterol in a membrane, thus causing lysis (of cells or fluorescent liposomes). If the QS21 first associates with cholesterol in liposomes it is no longer lytic towards cells or other liposomes. This requires a minimum ratio of 0.5:1 chol:QS21(w/w).

Cholesterol is insoluble in aqueous solutions and does not form a stable suspension. In the presence of phospholipids the cholesterol resides within the phospholipid bilayer which can form a stable suspension of vesicles called liposomes. To avoid the requirement to add phospholipids a soluble derivative was tried. Polyoxyethanyl-cholesterol sebacate is soluble in water at 60 mg/ml however even at a 2000 fold excess (w/w) over QS21 no reduction in the lytic activity of QS21 was detected.

### 1.4 Increased Stability of QS21 by Liposomes Containing Cholesterol

QS21 is very susceptible to hydrolysis at a pH above 7. This hydrolysis can be monitored by measuring the decrease in the peak corresponding to QS21 on reverse-phase HPLC. For example, FIG. 2. shows that at pH 9 and at a temperature of 37° C., 90% of QS21 is hydrolysed within 16 hours. If liposomes containing cholesterol are added to the QS21 at a ratio of 2:1 (chol:QS21 w/w) no hydrolysis of the QS21 is detected under the same conditions. If the ratio is 1:1 10% of the QS21 is degraded.

It is concluded that when QS21 associates with liposomes containing cholesterol it becomes much less susceptible to base-mediated hydrolysis. The hydrolysis product is described as having no adjuvant activity when given parenterally, hence vaccines containing QS21 have to be formulated at acidic pH and kept at 4° C. to maintain adjuvant composition. The use of liposomes may overcome this requirement.

### 1.5 Reactogenicity Studies

Mice injected in tibialis muscle with 5 μg QS21 (or digitonin) added to increasing quantities of liposomes (expressed in terms of μg cholesterol). Lytic activity is expressed as μg QS21 equivalent, which means that quantity of QS21 required to achieve the same hemolysis as the sample. Redness, necrosis and toxicity in the muscle at the site of injection were scored visually after sacrificing the mice.

| formulation | lytic activity μg | redness | necrosis | toxicity |
|---|---|---|---|---|
| QS21 + PBS | 5 | +++ | ± | +++ |
| QS21 + 1 μg chol (SUV) | 4 | +++ | + | ++++ |
| QS21 + 5 μg chol (SUV) | 0 | − | − | ± |
| QS21 + 25 μg chol (SUV | 0 | ± | − | + |
| SUV alone | 0 | − | − | − |
| digitonin | 5 | − | − | ± |
| PBS | 0 | − | − | − |

The data shows that when the lytic activity is abolished by the addition of liposomes containing cholesterol the toxicity due to the QS21 is also abolished.

### 1.6 Reactogenicity Intra-Muscularly in Rabbits

Values in U.I./L

| Experiment | Formulation | Day0 | hemolysis | Day1 | hemolysis | Day3 | hemolysis |
|---|---|---|---|---|---|---|---|
| Rabbit n°1 | | 1078 | ± | 8650 | | 1523 | |
| Rabbit n°2 | | 1116 | | 4648 | | 1435 | |
| Rabbit n°3 | QS21 50 μg | 660 | | 4819 | | 684 | |
| Rabbit n°4 | | 592 | | 5662 | | 684 | |
| Rabbit n°5 | | 3400 | | 7528 | | 1736 | |
| Mean | | 1369 | | 6261 | | 1212 | |
| SD | | 1160 | | 1757 | | 495 | |
| Rabbit n°6 | | 596 | | 1670 | | 460 | |
| Rabbit n°7 | | 540 | | 602 | | 594 | |
| Rabbit n°8 | QS21 50 μg | 611 | | 1873 | | 803 | |
| Rabbit n°9 | Chol in | 521 | | 507 | | 616 | |
| | SUV 50 μg | | | | | | |
| Rabbit n°10 | (1:1) | 1092 | ± | 787 | | 555 | |
| Mean | | 672 | | 1088 | | 606 | |
| SD | | 238 | | 636 | | 125 | |
| Rabbit n°11 | | 332 | | 344 | | 387 | |
| Rabbit n°12 | | 831 | | 662 | | 694 | |
| Rabbit n°13 | QS21 50 μg | 464 | | 356 | | 519 | |
| Rabbit n°14 | Chol in | 528 | | 720 | | 614 | |
| | SUV 150 μg | | | | | | |
| Rabbit n°15 | (1:3) | 1027 | | 568 | | 849 | |
| Mean | | 637 | | 530 | | 613 | |
| SD | | 285 | | 173 | | 175 | |
| Rabbit n°16 | | 540 | | 769 | | 745 | |
| Rabbit n°17 | | 498 | | 404 | | 471 | |
| Rabbit n°18 | QS21 50 μg | 442 | | 717 | | (4535) | |
| Rabbit n°19 | Chol in SUV | 822 | | 801 | | 925 | |
| | 250 μg | | | | | | |
| Rabbit n°20 | (1:5) | 3182 | ± | 2410 | | 960 | |
| Mean | | 1097 | | 1020 | | 775 | (1527) |
| SD | | 1175 | | 793 | | 224 | (1692) |
| Rabbit n°21 | | 321 | | 290 | | 378 | |
| Rabbit n°22 | | 660 | | 535 | | 755 | |
| Rabbit n°23 | PBS | 650 | | 603 | | 473 | |
| Rabbit n°24 | | 1395 | | (3545) | | (5749) | |
| Rabbit n°25 | | 429 | ± | 323 | | 263 | |

| | | | | | |
|---|---|---|---|---|---|
| Mean | 691 | 438 | (1059) | 467 | (1523) |
| SD | 419 | 155 | (1396) | 210 | (2369) |

The data shows that the addition of cholesterol-containing liposomes to the formulation significantly reduces the elevation in CPK (creatine phospho kinase) caused by the QS21, Since the CPK increase is a measure of muscle damage this indicates decreased muscle damage and is confirmed by the histopathology.

### 1.7 Binding of the Liposome-QS21 Complex to Alum

QS21 was incubated with neutral liposomes containing excess cholesterol as well as radioactive cholesterol and then incubated with alum (Al(OH)₃) in PBS. Alone, neither neutral liposomes nor QS21 bind to alum in PBS, yet negatively charged liposomes do. When together however, QS21 and neutral liposomes bind to alum. The supernatant contained neither QS21 (assayed by orcinol test) nor radioactive cholesterol.

This indicates that the QS21 has bound to the liposomes and permits the liposome-QS21 combination to bind to the alum. This may arise from a negative charge being imposed on the liposomes by the QS21, or to an exposure of hydrophobic regions on the liposomes. The results also imply that QS21 does not extract cholesterol from the membrane.

This indicates that compositions of the invention can be used in alum based vaccines.

### 1.8 Comparison of liposomal QS21/3D-MPL and Free QS21+3D-MPL for Antibody and CMI Induction

SUV were prepared by extrusion (EYPC:chol:3D-MPL 20:5:1). For MPL out, liposomes were prepared without 3D-MPL and 3D-MPL added as 100 nm particles.

QS21 was added prior to antigen. Chol:QS21=5:1 (w/w)

MLV were made by freeze-thawing SUV 3× prior to antigen addition.
To have the antigen entrapped, the antigen was added to SUV prior to freeze-thawing and QS21 added after freeze-thaw. Antigen encapsulation=5% in, 95% out.
-mice (balb/c for gD, B10BR for RTSs) were injected twice in the footpad.
gD is the glycoprotein D from Herpes Simplex virus. RTSs is the Hepatitis B surface antigen (HBsAg) genetically modified to contain an epitope from the Plasmodiium falciparum sporozoit.

ag = 10 μg RTSs

| | anti HBsAg Titres | | |
|---|---|---|---|
| | 15 days post boost | | |
| formulation | IgG1 | IgG2a | IgG2b |
| SUV/QS + 3D-MPL(out) + Ag | 1175 | 10114 | 71753 |
| MLV/QS + 3D-MPL(out) + Ag | 2247 | 11170 | 41755 |
| MLV/QS/3D-MPL(in) + Ag | 969 | 7073 | 18827 |
| MLV/QS/3D-MPL(in)/Ag(in) + Ag | 1812 | 2853 | 9393 |
| QS + 3D-MPL + Ag | 372 | 9294 | 44457 |
| Ag | <100 | <100 | <100 |
| SUV/QS + 3D-MPL(out) | <100 | <100 | <100 |
| MLV/QS/3D-MPL(in) | <100 | <100 | <100 |

ag = 20 μg gD

| | | CMI | |
|---|---|---|---|
| | anti-gD | IFN-g96 hr | IL2 48 hr |
| formulation | IgG | (pg/ml) | pg/ml |
| SUV/QS + 3D-MPL(out) + Ag | 2347 | 1572 | 960 |
| SUV/QS/3D-MPL(in) + Ag | 2036 | 1113 | 15 |
| MLV/QS + 3D-MPL(out) + Ag | 1578 | 863 | 15 |
| MLV/QS/3D-MPL(in) + Ag | 676 | 373 | 15 |
| MLV/QS/3D-MPL(in)/Ag(in) + Ag | 1064 | 715 | 15 |
| QS + 3D-MPL + Ag | 1177 | 764 | 15 |
| Ag | <100 | 567 | 44 |
| SUV/QS + 3D-MPL(out) | <100 | 181 | 15 |
| MLV/QS/3D-MPL(in) | <100 | 814 | 105 |

The data shows that SUV/QS+3D-MPL(out) induces high antibody titres at least as good as QS21+3D-MPL, as well as inducing IL2 a marker of cell mediated immunity, while quenching QS21 reactogenicity.
Additional results from a second experiment comparing QS21 and QS21 in the presence of cholesterol (SUV) in balb/c mice with HSV gD as antigen are shown below:

| | | | | Isotypes 7days post II | | | | | |
|---|---|---|---|---|---|---|---|---|---|
| | | IgG 7 post | IgG 14post | | | | | | |
| | | II | II | IgG1 | | IgG2a | | IgG2b | |
| Formulation | antigen | (GMT) | (GMT) | μg/ml | % | μg/ml | % | μg/ml | % |
| SUV/QS21 + MPL out | gD (5 μg) | 20290 | 16343 | 331 | 26 | 716 | 56 | 222 | 17 |
| SUV/QS21/MPLin | gD (5 μg) | 12566 | 10731 | 418 | 44 | 412 | 44 | 111 | 12 |
| QS21 + MPL | gD (5 μg) | 10504 | 10168 | 200 | 34 | 285 | 48 | 107 | 18 |
| SUV/QS21 + MPL out | none | 0 | 0 | 0 | 0 | 0 | 0 | 0 | 0 |
| QS21 | gD (5 μg) | 3468 | 4132 | 156 | 66 | 67 | 28 | 14 | 6 |
| SUV/QS21 | gD (5 μg) | 11253 | 11589 | 484 | 57 | 304 | 36 | 65 | 8 |

### 1.9 Comparison of gp120 Plus Liposomal MPL/QS21 with Free MPL/QS21

Liposomes=SUV containing MPL in the membrane

### Chol:QS21=6:1

Response was tested two weeks after one immunisation

| | | IFN-g | IL2 | IL5 |
|---|---|---|---|---|
| formulation | proliftn | ng/ml | pg/ml | pg/ml |
| SUV/MPL/QS21 + Ag | 12606 | 16.6 | 59 | 476 |
| MPL + QS21 + Ag | 16726 | 15.8 | 60 | 404 |

After second immunisation:

| | | IFN-g | IL4 | IL5 |
|---|---|---|---|---|
| formulation | proliftn | ng/ml | pg/ml | pg/ml |
| SUV/MPL/QS21 + Ag | 12606 | 135 | 0 | 250 |
| MPL + QS21 + Ag | 16726 | 60 | 0 | 500 |

The data shows that QS21 associated with cholesterol-containing liposomes and MPL induces Th1/Th0 response equal to MPL+QS21.
At this ratio of cholesterol to QS21, QS21 is non-toxic in rabbits (by CPK).

In a second experiment balb/c mice were immunised intra-footpad with gp120 in the presence of QS21 or QS21+SUV containing cholesterol. The cytotoxic T-lymphocyte activity in spleen cells was measured.

This demonstrates that QS21 alone induces CTL activity, and that QS21 in the presence of liposomes containing cholesterol induces CTL activity at least as good as, or better than, QS21 alone.

### 2. Vaccines

2.1 Formulation of HBsAg L*,S particles.

HBsAg L*,S particles may be formulated as follows:

10 μg of HBsAg L*,S particles/dose are incubated 1 h. at room temperature under agitation. The volume is adjusted using water for injection and a PBS solution and completed to a final volume of 70 μl/dose with an aqueous solution of QS21 (10 μg/dose). pH is kept at 7±0.5.

Similar formulations may be prepared using 1 and 50 μg of HBsAg L*,S and also using the HBsAg S antigen.

These formulations may be tested in the Woodchuck surrogate therapeutic model using Woodchuck HBV antigens as a model.

### Woodchuck Model

DQ QS21 (i.e. QS21/cholesterol or quenched QS21) may be tested in the woodchuck therapeutic model where animals are chronically infected with the virus. Specific woodchuck hepatitis virus vaccine may be add mixed with QS21 as such or DQ and with or without MPL and administered to the animals every months for 6 months.

Effectiveness of the vaccine may be assess through viral DNA clearance.

### 2.2 Guinea Pig Model (HSV)

### 2.2.1 Prophylactic Model

Groups of 12 female Hartley guinea pigs were either injected intramuscularly on day 0 and day 28 with the following formulations:

1st experiment:
gD 5 μg+QS21 50 μg+SUV containing 50 μg cholesterol
gD 5 μg+QS21 100 μg+SUV containing 100 μg cholesterol
gD 5 μg+QS21 50 μg+SUV containing 250 μg cholesterol
gD 5 μg+QS21 50 μg
2nd experiment:
gD 5 μg+MPL 12.5 μg+QS21 12.5 μg+SUV containing 62.5 μg cholesterol, or left untreated.

The animals were bled at 14 and 28 days after the second immunisation, and the sera tested for their gD-specific ELISA antibody titres.

Animals were then challenged intravaginally with 10⁵ pfu HSV-2 MS strain. They were scored daily from day 4 to 12 for evaluation of primary herpetic lesions. Scoring was as follows:

Vaginal lesions:

bleeding=0.5

redness for one or 2 days without bleeding=0.5

redness and bleeding for a day=1

redness without bleeding lasting at least 3 days=1

External herpetic vesicles:

<4 small vesicles=2

>=4 small vesicles or one big vesicle 4>=4 large lesions 8 fusing large lesions=16

fusing large lesions on all external genital area=32.

The results are shown in the table below:

### Prophylactic Model

Experiment 1 (chol refers to SUV containing cholesterol)

| | | PRIMARY DISEASE | | | | | | |
|---|---|---|---|---|---|---|---|---|
| | | Animal | Vaginal | External | | | | |
| | | without | lesions | lesions | | | Lesion | |
| | | lesion | incidence | incidence | PI | reduction | severity * | |
| n | FORMULATION | % | % | % | Index ** | vs Control | Median | n |
| 12 | gD/QS21 50 μg | 50 | 33 | 17 | 73 | 93% | 1.50 | 6 |
| 11 | gD/QS21 50 μg-chol 1/5 | 64 | 18 | 18 | 67 | 93% | 2.50 | 4 |
| 12 | gD/QS21 50 μg-chol 1/1 | 100 | 0 | 0 | 0 | 100% | — | — |
| 12 | gD/QS21 50 μg-chol 1/1 | 50 | 33 | 17 | 54 | 95% | 0.75 | 6 |
| 12 | UNTREATED | 25 | 0 | 75 | 996 | — | 55.00 | 9 |

### Experiment 2

| | | | | | PRIMARY DISEASE | | | | | | |
|---|---|---|---|---|---|---|---|---|---|---|---|
| | | Ab titres (GMT) | | | Animal | Vaginal | External | | | | |
| | | ELISA | | NEUTRA | without | lesions | lesions | | | Lesion | |
| | | day 14 | day 28 | day 28 | lesion | incidence | incidence | PI | reduction | severity * | |
| n | FORMULATION | post II | post II | post II | % | % | % | Index ** | vs Control | Median | n |
| 12 | gD/QS21/SUV/MPL | 47006 | 31574 | 449 | 58.33 | 33.33 | 8.33 | 37.50 | 94% | 1.00 | 5 |
| 12 | UNTREATED | <400 | <400 | <50 | 16.67 | 8.33 | 75.00 | 587.50 | — | 11.50 | 10 |

* Sum of the lesion scores for the days 4 to 12 post-infection (animals without lesion are not considered).
Lesion scores: no lesion (0), vaginal lesions (0.5 or 1), external skin vesicles (2, 4, 8 or 16)
** Primary infection index = SUM (Max.score i) × (Incidence %) ; with i = 0, 0.5, 1, 2, 4, 8 or 16

The table and graph show that in the prophylactic model, a very high level of protection against primary disease was induced upon immunisation with gD/MPL/QS21/SUV. Both the incidence of external lesions and the lesion severity appeared highly reduced in the group of animals immunised with gD/MPL/QS21/SUV.

### 2.2.2 Therapeutic Model

In the therapeutic model, female Hartley guinea pigs were first challenged with 10⁵ pfu HSV-2 MS strain. Animals with herpetic lesions were then randomly allotted to groups of 16.

On day 21 and day 42, they were either immunised with one of the following formulations:

gD+MPL 50 μg+QS21 50 μg+SUV containing 250 μg cholesterol,
gD+Al(OH)3+MPL 50 μg+QS21 50 μg, +SUV containing 250 μg cholesterol or left untreated.

They were monitored daily from day 22 to 75 for evaluation of recurrent disease. Scoring was as described for the prophylactic model. The results are shown in the table and graph below:

Therapeutic Model

| | | SEVERITY * | | DURATION ** | | EPISODE NBER *** | |
|---|---|---|---|---|---|---|---|
| | | | % reduction | | % reduction | | % reduction |
| n | FORMULATIONS | Median | vs Control | Median | vs Control | Median | vs Control |
| 16 | gD + MPL + QS21 + | 9.00 | 43% | 7.00 | 18% | 3.00 | 14% |
| | SUV | | | | | | |
| 15 | gD + Al(OH)3 + MPL + QS21 + SUV | 8.50 | 46% | 7.00 | 18% | 3.00 | 14% |
| 16 | Untreated | 15.75 | — | 8.50 | — | 3.50 | — |

* Sum of the lesion scores for the days 22 to 75 post-infection .
** Total days animals experienced recurrent lesions for the days 22 to 75 post infection
*** Recurrence episode number for the days 22 to 75 post infection. One episode is preceded and followed by a day without lesion and characterized by at least two days with erythema (score = 0.5) or one day with external vesicle (score>=2)
Immunotherapeutical treatment performed on days 21 and 42.

The results show that good levels of protection were also induced in the therapeutic model of HSV-2 infection. Immunization with gD/MPL/QS21/SUV with or without Alum had a marked effect on the median severity of recurrent disease. It also slightly reduced episode number and duration (see Table).

### Example 3

### Preparation of Vaccine Containing Alum, SUV, 3D-MPL and QS21

3.1 Method of Preparation of SUV

A mixture of lipid (such as phosphatidylcholine either from egg-yolk or synthetic) and cholesterol in organic solvent, is dried down under vacuum (or alternatively under a stream of inert gas). An aqueous solution (such as phosphate buffered saline) is then added, and the vessel agitated until all the lipid is in suspension. This suspension is then microfluidised until the liposome size is reduced to 100 nm, and then sterile filtered through a 0.2 μm filter. Extrusion or sonication could replace this step. Typically the cholesterol:phosphatidycholine ratio is 1:4 (w/w), and the aqueous solution is added to give a final cholesterol concentration of 5 to 50 mg/ml.

3.2 Antigen (1-500 μg, preferably 10-100 μg) is added to alum eg (aluminium hydroxide or aluminium phosphate) (100-500 μg) in water. The volume of water is chosen so that the volume of the final formulation is 500 μl. After incubating for 15-30 minutes, 50 μg of MPL is added in the form of small-particle MPL (WO94/21292). The MPL is left to adsorb onto the alum for 15-30 minutes at room temperature. 10-times concentrated phosphate buffered saline (1.5 M sodium chloride, 0.5M sodium phosphate pH 7.5) is then added in such a volume so as to render the final formulation isotonic. This formulation is incubated at room temperature for 15-30 minutes.

QS21 (50 μg) is then added to SUV (containing between 50 and 250 μg cholesterol). This mixture is added to the above alum/antigen/MPL/buffer mixture. If required a bacteriostatic such as thiomersal is added (50 μg).

### Example 2

The following Table shows the binding of QS21 to alum in the presence and absence of liposomes containing 25% (w/w) in dioleoyl phosphatidylcholine, and using a five-fold excess of cholesterol over QS21.

| | | μg QS21 |
|---|---|---|
| Formulation | SUV | bound |
| 500 μg Alum + 50 μg QS21 | 0 | <10 |
| 500 μg Alum + 50 μg QS21 | 250 μg chol + 1 mg DOPC | >40 |

In order to increase the binding of QS21 to alum, the quantity of liposomes can be decreased. This decreases the cholesterol:QS21 ratio, however it has been shown that the QS21 remains non-toxic for cholesterol:QS21 ratios of 1:1 and greater. Table 2 shows that if the quantity of alum is decreased (from 500 μg to 100 μg) the quantity of QS21 that is bound decreases significantly, and the quantity of MPL that is bound also decreases. By adding less liposomes, yet maintaining a cholesterol:QS21 ratio of 1:1 or greater, increased quantities of QS21 and MPL can be bound to the alum.

| Formulation | Chol/QS21 | μg QS21 bound | μg MPL bound |
|---|---|---|---|
| 500 μg alum + 50 μg | 5/1 | 42 | >48 |
| QS21 + 50 μg MPL | | | |
| 100 μg alum + 50 μg | 5/1 | 17 | >40 |
| QS21 + 50 μg MPL | | | |
| 100 μg alum + 50 μg | 2/1 | 30 | >45 |
| QS21 + 50 μg MPL | | | |
| 100 μg alum + 50 μg | 1/1 | 40 | >45 |
| QS21 + 50 μg MPL | | | |

### Example 5

The adjuvant effect of a combination of antigen (gD2t from Herpes Simplex Virus-2—expressed in CHO cells and comprises 283 amino acid from the mature N-terminal of the mature glycoprotein) with MPL and QS21 in combination with liposomes was tested with and without alum. The formulations were tested in African Green Monkeys.

African Green Monkeys were immunised twice (0, 28 days) with 20 μg gD2t plus 50 μg MPL plus 50 jag QS21 with or without liposomes (250 μg cholesterol plus 1 mg DOPC) and with or without 500 μg alum. On day 42 the immune response was analysed.

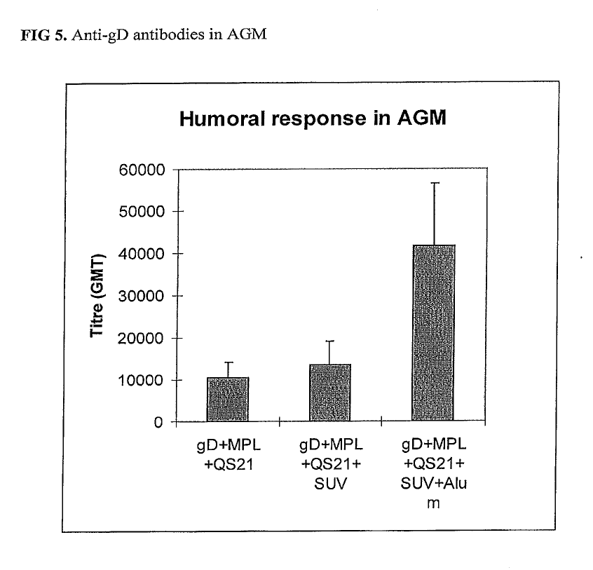

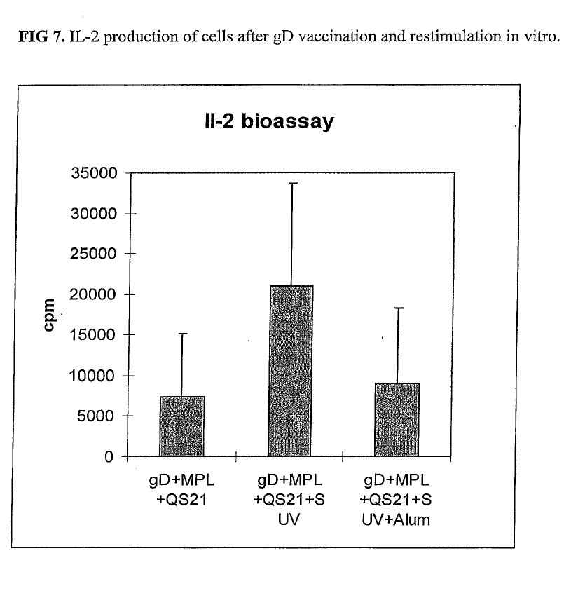

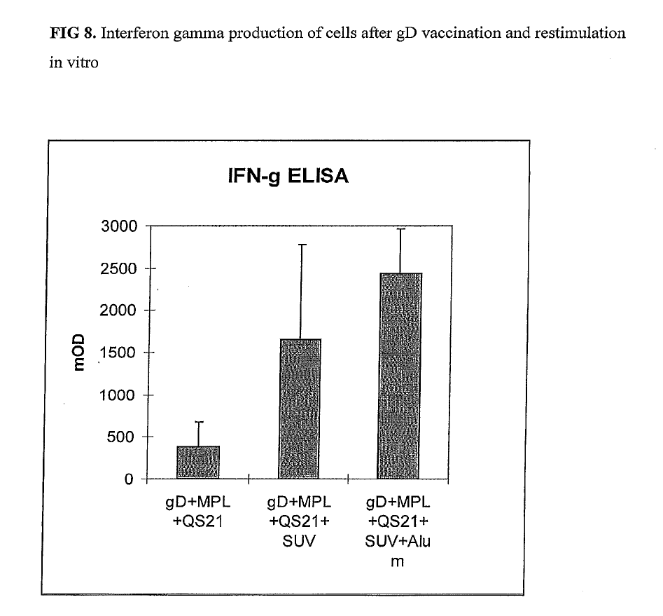

The results are outlined below in FIGS. 5 to 8.

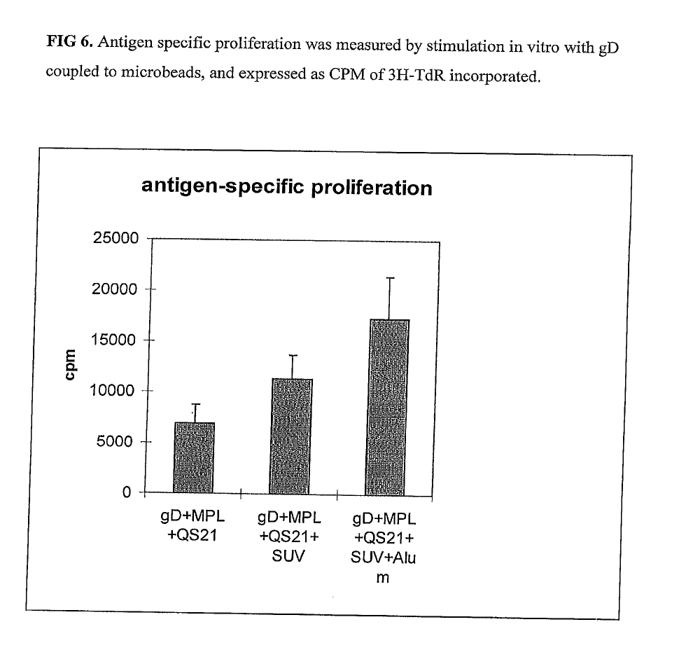

The humoral response was measured as IgG against the gD protein. FIG. 5 shows that the combination of MPL+QS21+SUV+alum induced higher titres than in the absence of alum. FIG. 6 shows that the formulation of the invention provided the superior antigen specific proliferation.

The data shows that incorporation of aluminium hydroxide in vaccine formulations containing MPL and QS21 and SUV enhances both the humoral and cellular responses. This is an unexpected finding since it is generally accepted that aluminium as an adjuvant tends to favour Th2 type responses, yet the results presented here demonstrate that the response contains a significant Th1 component which is not depressed by the addition of alum.

The formulation containing MPL and QS21 and SUV and alum is non-toxic and highly immunogenic.

### Example 6

### Production of RSV FG CHO Cell Derived Proteins

The plasmid pEE14-FG contains a chimeric construct comprising of a fusion between amino acid sequences of F (1-525) and G (69-298) and was received from a collaboration with A. BOLLEN (ULB/CRI, Belgium). This FG fusion protein contains a total of 755 amino acids. It starts at the N-terminal signal sequence of F and lacks the C-terminal transmembrane domain (525-574)—anchor domain—of F glycoprotein. Then, followed the extracellular region of G glycoprotein, without the amino-terminal region that contains the Signal/Anchor domain of G, a typical class II glycoprotein.

The pEE14-FG expression plasmid was generated by the insertion of the FG coding sequence from pNIV2857 (A. Bollen, ULB/CRI, Belgium) as an Asp7181 (blunt) 5′—HindIII (blunt) 3′ restriction fragment (2188 bp) into the SmaI site of pEE14 (Celltech). A Kozak sequence in lieu of the FG start ATG was generated into the pNIV 2857 construction as follows:

```
pEE14---ccc gtacc ATG GAG -----x-----CAG TAG aagct ggg ---pEE14
              (SmaI) Met1 Gln(298)Stop
                      Asp718I(klenow) HindIII(klenow)
                              5′F(1-525)-------x--------G(69-298)3′
```

The F sequence in pEE14-FG is from SS2 RSV strain, and was kindly made available by Dr. PRINGLE as a cDNA construct in a Vaccinia vector (Baybutt and Pringle, J. Gen. Virol., 1987, 2789-2796). The G sequence is from A2 RSV strain and was generated from a recombinant G Vaccinia virus obtained from Dr. G. WERTZ (Alabama, USA).

### CHO K1 Transfection and Stable FG Protein Expression.

CHO K1 cells derived from MCB 024M (Celltech) were transfected with 20 ug of pEE14-FG plasmid DNA twice CsCl purified using the Ca-phosphate-glycerol transfection procedure. Cell clones were selected according to the procedure of the GS (glutamine synthetase) expression system (Crocett et al BioTechn., 1990, Vo18, 662) and amplified in the presence of 25 micro molar methionine sulphoximine (MSX) in G MEM medium containing no glutamine and supplemented with 10% dialyzed FBS (Foetal Bovine Serum). Following transfection, 39 MSX resistant clones were screened in 24-well plates and their supernatants were tested for secretion of the FG fusion protein. All transfectants proved to be positive for F antigen expression using a specific ‘Sandwich’ ELISA assay (i.e. rabbit polyclonal anti FG serum/Antigen/mAB19). Monoclonal antibody 19 recognises a conformational F1—epitope and is neutralising.

The 3 best FG-producer clones (n° 7, 13 and 37) were single-cell subcloned in a limiting dilution assay using 0.07 cells per well in a 96-well plates. A total of 59 positive subclones were obtained and the 16 best FG-producers were further characterised by western blot and ELISA. Again, the 8 best FG-subclones were further amplified and their FG expression was evaluated in presence and absence of sodiumbutyrate (2 mM) or DMSO (1 or 2%). Six subclones were amplified and cell vials were made and stored at −80° C. and liquid N2. Finally, the 3 best FG-subclones were selected. These are CHOK1 FG °7.18, °13.1, and °37.2.

Westernblot analyses (non-reducing conditions) with monoclonal mAB 19 indicated a major band of FG at about 135 kDa. The purified FG protein from recombinant Baculovirus FG infected cells (UPJOHN) appeared as major broad bands at +/−100 kDa and other bands at +/−70 kDa under similar blot conditions.

The addition of Sodium butyrate in CHO-FG cell culture medium increased the expression level of FG 3 to 12 fold depending on the subclone and cell culture growth conditions. In particular, subclone CHO-FG 13.1 expressed 8-10 fold more FG protein in the presence of butyrate (WB/ELISA).

Expression level determination was performed by ELISA (mAB19 or MoAb AK13) using purified FG baculo protein as standard, as well as by western blot analysis using serial dilution.

Depending of the ELISA assay and cell culture conditions, the expression level of CHO-FG 13.1 is 5-12 ug of FG/ml after treatment with butyrate. Under accumulation conditions and medium replacements (3 to 5 days) yields of 16 to 28 ug of secreted FG protein/ml were obtained.

CHOK1 FG 13.1 cell line was adapted to grow in suspension and serumfree (S/SF) conditions using a proprietary growth medium. Cell line CHO-FG 13.1 S/SF grown in a medium without butyrate expressed similar yields as the parental adherent cell line grown in medium with butyrate. The addition of butyrate to CHO-FG 13.1 S/SF media has little effect on production of FG (1.5 to 2 fold increase).

Long term expression evaluation and preliminary genetic characterisation showed that CHO-FG 13.1 and the S/SF adapted 13.1 cell line were stable, contained intact FG expression cassettes giving rise to one single mRNA band of about 3000 nucleotides long (Southern and Northern analyses). The CHO-FG clone 13.1 S/SF was further used for production of FG antigen.

The use of alum/MPL/QS21/SUV for the enhancement of the immune response in African Green Monkeys towards the FG protein from RSV (Respiratory Syncytial Virus).

The FG protein (fusion protein containing the F- and G-proteins from RSV) was expressed in CHO cells and purified. 20 μg of the purified protein was adsorbed on alum (500 μg) to which monophosphoryl lipid A (MPL: 50 μg) was added. After incubating 30 minutes at room temperature, phosphate buffered saline was added. Then either SUV alone or a mixture of SUV and QS21 (50 μg QS21, SUV containing 250 μg cholesterol and 1 mg DOPC) were added. African green monkeys were injected three times with these formulations, or with FG on alum alone or FG mixed with MPL, SUV and QS21 in the absence of alum.

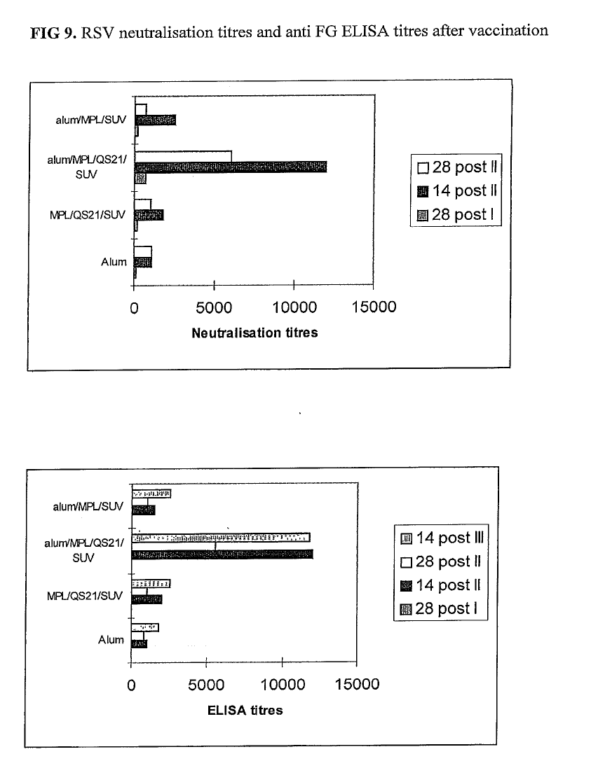

FIG. 9 below show the RSV neutralising titres and the FG-ELISA titres obtained for each formulation. It is clear that the group alum/MPL/QS21/SUV induces the highest titres.

### Example 7

### Comparison of QS21/SUV Containing Formulations with Alum Formulation of Hepatitis B Vaccine Containing SL* Antigen

### Introduction

SL* was produced in accordance with the procedure set out in European Patent application No. 414374.

An immunogenicity study was conducted in Balb/c mice to compare the humoral responses induced by QS21-SUV containing formulations in presence or not of Al(OH)3. MPL dose was 5 μg, QS21 5 μg, SUV contained 25 μg cholesterol and 100 μg DOPC.

The experimental protocol is described in Material and Methods.

Briefly, mice were immunised intramuscularly in the leg twice at 4 weeks interval with SL* vaccines containing vehicle, immunostimulants or combinations of both. Anti-HBs humoral responses (IgG and isotypes) were analysed.

The following groups were included in the study:

1. SL* (2 ug) Al(OH)3 (50 ug)

2. SL* (2 ug) Al(OH)3 (50 ug)/MPL/QS21-SUV

3. SL* (2 ug) Al(OH)3 (50 ug)/QS21-SUV

4. SL* (2 ug) MPL/QS21-SUV

### Results

Humoral responses were measured by Elisa as described in Material and Methods. Two time points were analyses: 28 days after the first injection (28 post I) and 14 days following the booster injection (14 post II).

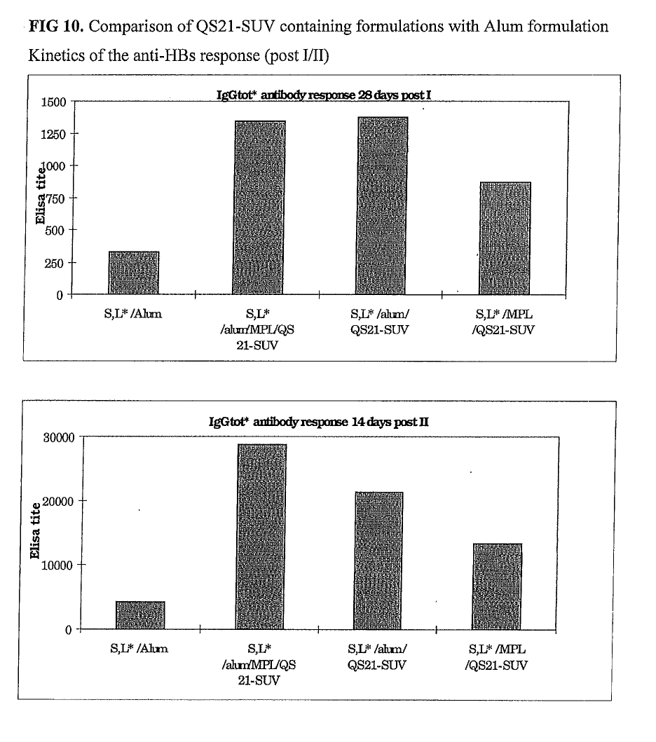

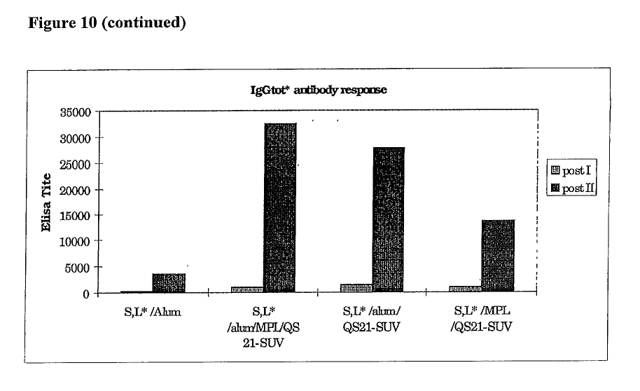

Post I and post II anti-HBs response analysed on pooled sera are presented in FIG. 10.

These data show that in primary response, comparable antibody titers are induced by all formulations containing QS21-SUV while a weaker response is observed when Al(OH)3 alone is used.

In secondary response, the lowest antibody response was also induced by Al(OH)3 containing vaccine. However, all formulations containing QS21-SUV did not behave the same way.

The two formulations containing Al(OH)3 QS21-SUV (+/−MPL) induced the strongest antibody response (2× higher than MPL/QS21-SUV).

Although no statistical analysis has been performed, results on individual sera confirm this observation.

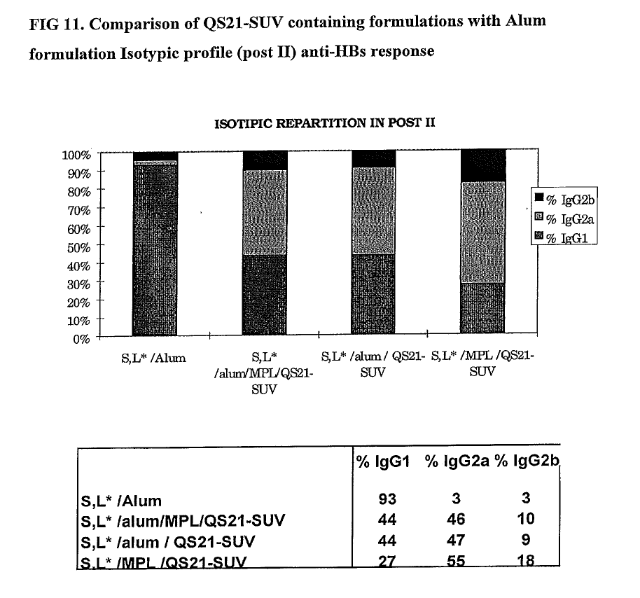

The combination of Al(OH)₃ and QS21-SUV (+/−MPL) also qualitatively affects the immune response as shown by the isotypic profile of the humoral response (FIG. 11). Al(OH)3 induces a clear TH2 type of immune response (only 3% IgG2a) whereas Al(OH)3/QS21-SUV (+/−MPL) formulations induce up to 46% IgG2a.

### Conclusion

The combination of Alum with QS21-SUV (+/−MPL) induces higher antibody titers than formulations containing vehicle or immunostimulants alone.

### Material and Methods

### Immunisations

10 groups of 5 female Balb/c mice (6-8 weeks) were immunised intramuscularly in the leg (gastrocnemien) twice at 4 weeks interval with 500 vaccine containing 2 μg SL* formulated in Al(OH)3(50 ug equivalent A13+), Al(OH)3/QS21-SUV, Al(OH)3/MPL/QS21-SUV, MPL/QS21-SUV. A dose of 5 ug of immunostimulants was used.

Animals were bled on day 28 (28 post I) and 42 (14 post II) for antibody determination by Elisa.

### Formulations

Components batches used.

### Formulation Process

SL* (2 ug) is adsorbed or not for 15 min on 50 ug of water diluted Al(OH)3.

If needed, 5 ug of MPL is added to the preparation as a suspension of 100 nm particles (MPL out) for 15 min. If needed, ten fold concentrated buffer is added before adding 5 ug of QS21 mixed with liposomes in a weight ratio QS21/Cholesterol of 1/5.

Thiomersal is added to the formulations 15 min after QS21/SUV addition.

Formulations containing QS21-SUV are buffered with PBS pH 7.4 and the others are prepared in PBS pH 6.8

### Serology

Quantitation of anti-HBs antibody was performed by Elisa using HBs (Hep286) as coating antigen. Antigen and antibody solutions were used at 50 ul per well. Antigen was diluted at a final concentration of 1 ug/ml in PBS and was adsorbed overnight at 4° c. to the wells of 96 wells microtiter plates (Maxisorb Immuno-plate, Nunc, Denmark). The plates were then incubated for 1 hr at 37° c. with PBS containing 1% bovine serum albumin and 0.1% Tween 20 (saturation buffer). Two-fold dilutions of sera (starting at 1/100 dilution) in the saturation buffer were added to the HBs-coated plates and incubated for 1 hr 30 min at 37° c. The plates were washed four times with PBS 0.1% Tween 20 and biotin-conjugated anti-mouse IgG1, IgG2a, IgG2b or a mix of the three antibodies (Amersham, UK) diluted 1/1000 in saturation buffer was added to each well and incubated for 1 hr 30 min at 37° c. After a washing step, streptavidin-biotinylated peroxydase complex (Amersham, UK) diluted 1/5000 in saturation buffer was added for an additional 30 min at 37° c., Plates were washed as above and incubated for 20 min with a solution of o-phenylenediamine (Sigma) 0.04% H2O2 0.03% in 0.1% tween 20 0.05M citrate buffer pH4.5. The reaction was stopped with H2SO4 2N and read at 492/620 nm. ELISA titers were calculated from a reference by SoftmaxPro (using a four parameters equation) and expressed in EU/ml.

### Example 8

### Production of QS21 and Cholesterol Containing Liposomes

40 mg of DOPC (dioleoyl phosphatidylcholine) and 10 mg of cholesterol was solubilised in choloroform and evaporated into a thin film by vaccuum dessication. The film was resuspended with 1 ml of PBS at pH 7.4 (10 mMPO₄, 150 mM NaCl) to form multilamellar liposomes (MLV). The MLV were the microfluidised for 5 minutes (Microfluidiser M110S, which corresponds to 37.5 cycles), to form small unilamellar vesicles (SUV). 200 μl of SUV were then mixed with 200 μl of QS21 (stock of 1 mg/ml) which corresponds to a ratio of (5:1 cholesterol:QS21 w/w). The adjuvants were confirmed as liposomes (with the QS21 forming stable pores in the surface of the membrane) and not the cage-like ISCOM structure.

The resulting adjuvant formulation had a mean size of 114 nm and 115 nm as measured using Photon correlation spectroscopy on the Malvern Zetasizer 4000, Laser wavelength 633 nm, Laser power 10 mW, Scattered light detected at 90° C., T° 25-26° C., Duration: automatic determination by the software, 2 consecutive measurements of a X100 dilution of sample, Size distribution by the CONTIN method, z average diameter by cumulants analysis.

The same process is also used to insert 3D-MPL into the vesicle membrane, by adding 3D-MPL to the DOPC and cholesterol in the cholorform; and resuspending the film in PBS either at pH 7.4 or pH 6.1 (PBS 50 mMPO4 10 mM NaCl). These adjuvants may also be adsorbed onto aluminium hydroxide or aluminium phosphate.
Vaccines may be formulated with these adjuvants by the admixture of antigen, or by incorporating it into the vesicle (adding it into the choloroform/lipid mixture).

### Example 9

### Human Clinical Trial

A double-blind, randomised Phasel/II study was conducted to compare the capacity of various adjuvant formulation containing Hepatitis B surface antigen (HbsAg) to induce Cytotoxic T Lymphocytes (CTL) in healthy adult volunteers. Groups of roughly 50 subjects were included in the study. The vaccinees received 3 intramuscular injections, the first at time 0, and two boosting doses at the 1 month and 10 month time points.

### Vaccine Group 1

20 μg HBsAg plus oil in water emulsion adjuvant containing QS21 and 3D-MPL (100 μg 3D-MPL, 100 μg QS21, 250 μl of a squalene and α-tocopherol oil emulsion (for details of formulation see the description in U.S. Pat. No. 6,146,632)) in a total volume of 0.5 ml.

### Vaccine Group 2

20 μg HBsAg plus a liposomal adjuvant comprising the saponin QS21 and 3D-MPL (SUV-containing 50 μg 3D-MPLin the membrane, 50 μg QS21, and 250 μg cholesterol (chol:QS21 5:1 w/w)) in a total volume of 0.5 ml.

### Vaccine Group 3

20 μg HBsAg plus 50 μg 3D-MPL, 50 μg QS21, 50 μl oil in water emulsion (same as vaccine group 1) containing 100 μg cholesterol (chol:QS21 2:1 w/w) administered in a total volume of 0.5 ml.

CTL analysis was performed on cells from pre and post II (14 days post II) plasma samples. The intensity of the CTL response was assessed through the measure of percentage of lysis on target ratios (90:1, 30:1, 10:1).

The percentage of lysis was been calculated as follows:

% lysis=((cpmₛₐₘₚₗₑ)−GM(cpm_{spontaneous release}))/(GM(cpm_{maximum release})−GM(cpm_{spontaneous release}))

where GM=geometric mean.

The analysis of the primary endpoint was be based on the % of specific lysis. It is defined as: % specific lysis=GM(% lysis_{relevant peptides})−GM(% lysis_{irrelevant peptides}).

Values smaller than 1 were considered to be 1.

### Results

Results are expressed as number of responders and % responders using the following definition of responder:

If % lysis PRE<cut-off and % lysis POST>=cut-off=>responder

If % lysis PRE>=cut-off and % lysis POST−% lysis PRE>=10%=>responder

Otherwise, non responder

Number of CTL responders

| | | | Group 2 | | Group 3 | |
|---|---|---|---|---|---|---|
| | Group 1 | | (N = 46) | | (N = 41) | |
| | (N = 43) | | | % of | | % of |
| Ratio | responders | % of N | responders | N | responders | N |
| 10/1 | 2 | 4.7 | 3 | 6.5 | 4 | 9.8 |
| 30/1 | 6 | 14.0 | 11 | 23.9 | 8 | 19.5 |
| 90/1 | 7 | 16.3 | 17 | 37.0 | 12 | 29.3 |

p-values were calculated (see following Table) and showed statistically significant differences in CTL induction between Group 2 and Group 1.

| | Group 2 | Group 3 |
|---|---|---|
| Group 1 | 0.0493 | Not Significant |
| Group 2 | — | Not Significant |
| Group 3 | Not Significant | — |

### Conclusions

The adjuvants of the present invention, containing a saponin (QS21) and a sterol (cholesterol), induce high amounts of Hepatitis virus specific CTL in humans. The cholesterol containing adjuvants, together with the QS21, induced significantly better CTL responses that those QS21 containing adjuvants that did not contain cholesterol. Liposomal adjuvants (group 2) of the present invention tended to induce higher amounts of CTL responses than other saponin (QS21) and sterol (cholesterol) containing formulations.

In addition to the CTL responses, the cholesterol containing formulations were reported by the vaccinees to be less painful and induce fewer side effects of reduced severity compared to those reported for the non-cholesterol group 1. It is accepted in the scientific community that anti-HBs Ig titers superior or equal to 10 mIU/ml confer protection against HepB infection. This protective titer is usually evaluated following 3 injections with Hepatitis B vaccine (for example Engerix B™). In this trial all of the subjects vaccinated in groups 1, 2 and 3 had anti-HBs titers >10 mIU/ml following 2 injections (100% seroprotection for the three groups).

What is claimed is:

1. A method of reducing the reactogenicity of purified QS21 containing adjuvant formulations comprising adding sterol to the adjuvant formulation in excess of the QS21 (weight/weight).

2. The method of claim 1, wherein the sterol is in excess to the QS21 at a QS21:sterol ratio of up to 1:100 (w/w).

3. The method of claim 2, wherein the ratio of QS21:sterol is at least 1:2 (w/w).

4. The method of claim 3, wherein the ratio of QS21:sterol is 1:5 (w/w).

5. The method of claim 1, wherein the sterol is cholesterol.

6. A method of stabilising purified QS21 against alkali mediated hydrolysis in purified QS21 containing adjuvant formulations, comprising adding sterol to the adjuvant formulation in excess of the QS21 (weight/weight).

7. The method of claim 6, wherein the sterol is in excess to the QS21 at a QS21:sterol ratio of up to 1:100 (w/w)

8. The method of claim 7, wherein the ratio of QS21:sterol is at least 1:2 (w/w).

9. The method of claim 8, wherein the ratio of QS21:sterol is 1:5 (w/w).

10. The method of claim 2, wherein the sterol is cholesterol.
